(19) United States
(12) Patent Application Publication
(10) Pub. No.: US 2008/0200802 A1
(43) Pub. Date: Aug. 21, 2008

(54) PLATFORM FOR DETECTION OF TISSUE CONTENT AND/OR STRUCTURAL CHANGES WITH CLOSED-LOOP CONTROL IN MAMMALIAN ORGANISMS

(75) Inventors: Naresh C. Bhavaraju, San Diego, CA (US); Darrel D. Drinan, San Diego, CA (US); Carl F. Edman, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PhiloMetron, Inc., San Diego, CA (US)

(21) Appl. No.: 11/952,924

(22) Filed: Dec. 7, 2007

Related U.S. Application Data
(60) Provisional application No. 60/873,844, filed on Dec. 7, 2006.
(60) Provisional application No. 60/918,534, filed on Mar. 16, 2007.

(51) Int. Cl. A61B 5/053; A61B 5/055

(52) U.S. Cl. 600/426; 600/425; 600/547

(57) ABSTRACT

Aspects include methods and apparatuses for effecting change over time in one or more measured regions of a body using a plurality of data sets obtained by analysis of applied signals to said region and effecting a change in treatment protocol. The method may include transmitting and receiving one or more of electromagnetic wave signals, applied acoustic wave signals and electrical signals transmitted through or reflected off of a portion of the measured body region. Some aspects may include determining a change in tissue structure, or a change in tissue content.

### CROSS-REFERENCE TO RELATED APPLICATIONS

This application claims priority under 35 U.S.C. 119(e) to U.S. Provisional Application No. 60/873,844, filed on Dec. 7, 2006, entitled PLATFORM FOR DETECTION OF CONTENT AND/OR STRUCTURAL CHANGE OF TISSUE WITH CLOSED-LOOP CONTROL IN MAMALLIAN ORGANISMS, and U.S. Provisional Application No. 60/918,534, filed on Mar. 16, 2007, entitled IMPLANTED PLATFORM FOR DETECTION OF TISSUE CONTENT AND/OR STRUCTURAL CHANGES WITH CLOSED-LOOP CONTROL IN MAMMALIAN ORGANISMS. These applications are incorporated by reference in their entirety.

### BACKGROUND OF THE INVENTION

1. Field of the Invention

The invention relates to medical diagnostic equipment. In particular, the invention relates to monitoring systems to assess content and/or structure of tissue.

2. Description of the Related Art

Ultrasound is one common method of non-invasively measuring surface or subsurface tissue structures changes. Ultrasound employs the use of pulsed acoustic/sound waves and by interpretation of the reflected wave, imaging or other representations of subsurface structures can be made. However, the continuous or near continuous use of ultrasound devices on body is not readily feasible due to the need for contact between the ultrasonic head and the body skin or other body structure. Such contact is customarily made by use of gels which degrade or are lost over time, thereby limiting the effective on-body lifetime of ultrasonic devices in any one point. In addition, ultrasound cannot be used to measure changes beyond a certain depth inside the tissue as it is absorbed by the tissue and particularly hone.

### SUMMARY OF THE INVENTION

An aspect provides a method of periodically or continuously interrogating a tissue region of interest to determine if the content (e.g., a hydration state) or structure of said tissue has changed over time, or is different than a baseline “signature” stored in memory. Various forms of electromagnetic waves, acoustic waves or other forms of energy can be compared to the baseline with the use of a comparator for determination of regional changes over time of body structures or their content of mammalian bodies. The changes may be on the surface, beneath the surface or span the surface to the subsurface of the measured body region. For instance, such changes may include movement wherein internal structures or portions of structures change due to positional, contractile, etc. movement. In some embodiments, for example, measurements directed to tissue fluid content allow the determination of change, e.g. regional tissue fluid change, associated with fluid accumulation that may eventually lead to pulmonary edema or other disease states, unless therapeutic intervention occurs.

In one embodiment of the invention, electromagnetic waves based upon ultra wideband (UWB) radar technology are used. In alternate embodiments of the invention, bands of one or more other frequencies may be applied to the body for the determination of change. Alternate embodiments of the invention would use acoustic waves, light or electrical energy of any frequency. The measured values arising from a shift in the signal (electromagnetic or acoustic or other) are incorporated into a data set representing the status of the region at a certain time point. These data sets may then be stored for future comparison to data sets of measurements taken at other times to determine change in the properties or characteristics of a tissue or region being measured.

In another aspect of the invention, circuitry, a power supply and a transceiver for delivering and receiving the electromagnetic, optical, acoustic waves or other energy forms, e.g. signals, for construction of the measurement data set are contained either wholly or in part in a structure, e.g. by a patch or a unit, fully or partially affixed to or implanted within the body. In an alternate embodiment of the invention, the circuitry and transceiver for measuring signals are contained in a structure not affixed to or implanted in the body. The non-affixed/implanted structure may be hand held or otherwise supported to permit the measurement activity. Such supports may include inclusion of one or more devices into fixed structures present in the living space of measured subject, e.g. within beds, closets, bathrooms, etc., thereby permitting unobtrusive measurements to be obtained periodically without disruption of lifestyle or activities. In still other embodiments of the invention a portion of the measurement device may be located beneath the skin while the other aspects of the device either reside on the outside of the skin and/or project through the skin, e.g. are transcutaneous in nature.

The measured values arising from a shift in the signal (electromagnetic or acoustic or other) are incorporated into a data set representing the status of the region at a fixed time point. These data may then be stored for future comparison to data sets of measurements taken at other times to determine change in the properties or characteristics of a tissue or region being measured.

In some embodiments of the invention, guidance for placement of measurement devices and/or location of measurements activities may be utilized to aid determination of location of specific regions or targets of measurements. Guidance may be provided in the form of comparative mapping of body locations relative to either previous measurements and/or anatomical locations. Such anatomical locations may utilize anatomical landmarks and/or active or passive fiducial marks or devices.

The measurement devices may store information relating to the measurement event, e.g. time, or signal data, for later retrieval and analysis. In addition, the measurement devices may have in part or in whole comparators allowing processing of raw data, e.g. mathematical transforms, for the purpose of facilitating storage, transmittal or display of the signal data.

In one embodiment of the invention, the electromagnetic data sets are combined and utilized by the comparator with other physiological measurements, e.g. optical, electrical or mechanical, to provide greater insight into changes of the body region being measured. These other physiological measurements may include, but are not limited to, temperature, body weight, bioelectric impedance, or optical, e.g. infrared, measurements. In a preferred form of the invention, such measurements may include measurement of heart rate and/or heart waveform activity, e.g. electrocardiograms and values derived therefrom, or other measurements of body parameters, e.g. EEG or FMG. Such measurements may also include determination and possible alerts associated with abnormal body functionalities, e.g. atrial fibrillation. In other embodiments of the invention, the data set may be combined and used by the comparator with other measurements of physiological status, including but not limited to, nutritional and/or medical history, subjective responses to questions, diagnostic test results, e.g. blood composition analysis or urinalysis, to provide fuller assessment of possible changes in the region being measured.

In still other embodiments of the invention, one or more of the applied signals, e.g. radio frequency, optical or impedance, may be utilized for purposes of identification either of applied materials or devices on or within the mammalian body and/or for the purpose of identification of the mammalian body. This identification may be useful for numerous reasons, including but not limited to, ensuring correct management of applied therapies, or the tracking of devices and/or persons. Either existing structures within the body or applied materials may supply features necessary to ensure the correct level of identification or alternatively, additional markers or structures may be added for this purpose.

In some embodiments of the invention, the collected data sets may be transmitted by wireless or wired means to one or more data collection units. In certain embodiments of the invention where the measurement device is fully or partially implanted, the body or body structures may serve as the antenna or communication structure for the electrical, radiowave, acoustic or other suitable communication method. Upon reception of one or more data sets, a data collection unit may display the data set(s) and/or perform comparator activities upon the received data set and display the results of this activity. Such data collection units may collect data measurement sets from one or more measurement devices. Multiple measurement devices and/or measurement components, e.g. electrode or UWB antenna arrays, implanted or not implanted, may be used in combination to provide an image of tissue content or structural change. In addition, such data collection units may be used to display data set values, or mathematical transforms of said data sets, including trending and combinations with other sensor or input data. In yet other embodiments of the invention, said data sets or mathematical transforms of said data sets may be relayed to yet other data collection units or remote data management systems for data storage, display or additional analysis, e.g. population based or group trend analysis.

This invention is related, in part, to the methods and devices described in the following US patent and patent applications: U.S. Pat. No. 7,044,911 GATEWAY PLATFORM FOR BIOLOGICAL MONITORING AND DELIVERY OF THERAPEUTIC COMPOUNDS, US 20050070778 HYDRATION MONITORING, US 2006/0052678 MONITORING PLATFORM FOR WOUND AND ULCER MONITORING AND DETECTION, US 2006/0058593 MONITORING PLATFORM FOR DETECTION OF HYPOVOLEMIA, HEMORRHAGE AND BLOOD LOSS, U.S. Provisional filing Ser. No. 60/837,423 PLATFORM FOR DETECTION OF HYDRATION AND/OR STRUCTURAL CHANGE IN MAMMALIAN ORGANISMS, and U.S. Provisional filing Ser. No. 11/837,357 PLATFORM FOR DETECTION OF TISSUE STRUCTURE CHANGE, which are incorporated herein by reference in their entirety.

### BRIEF DESCRIPTION OF THE DRAWINGS

FIG. 1 is a schematic representation of an embodiment of a measurement device.

FIG. 2 is an illustration of a body limb section with a UWB device such as, for example, the device illustrated in FIG. 1, affixed to a surface of tissue.

FIG. 3 is an illustration of a slowing effect of a 120 ps UWB pulse due to fluid change in tissue.

FIG. 4 is a block diagram of a system for effecting change over time in accordance with one embodiment of the invention.

### DETAILED DESCRIPTION OF THE PREFERRED EMBODIMENT

There exists a need for an accurate, objective and convenient monitoring system to assess change in either the content, movement and/or structure of tissue within mammalian organisms. One area of this need is for the accurate assessment of fluid and/or tissue changes associated with the accumulation of fluid leading to disease states, e.g. pulmonary edema in congestive heart failure patients. Assessment of change in status may permit more effective therapeutic interventions and therapies to be applied in response to one or more of the measured change in status or allow more effective management of health-related conditions.

Alternative methods for measuring subsurface structures and content exist that utilize electromagnetic, infrared, acoustic and/or electrical waves. An electromagnetic form can be as ultra wideband micro impulse radar. This electromagnetic measurement may utilize time shifts of all or a portion of the transmitted wave to provide information regarding the tissue content or structure. The reflectance of these waves likewise provides information regarding internal structures. However, in its most common embodiment, such measurements are employed to evaluate movement of structures, rather than change in the composition of these structures. For example, McEwan (U.S. Pat. No. 5,573,012) teaches the use of pulse-echo radar in repetitive mode to measure heart motion. Likewise, Sharpe, et al., teach the use of radio frequency waves for non-contact measurement of movement of the surface of the body from which respiration and heart rate may be determined (U.S. Pat. No. 4,958,6380).

In an alternative use of radiowaves, Bridges (U.S. Pat. No. 5,829,437) teaches the use of backscattered radiowaves reflected by change in dielectric constant resulting from the differential composition of tissues to detect abnormalities, e.g. tumors, in tissue. However, Bridges does not teach the use of comparative measurements over time that would permit the automatic determination of a change in the tissue status, e.g. the recovery from or growth of tumors and the rate of the growth or recovery due to treatment. What is needed is a method utilizing a plurality of measurements to the allow determination of change in either tissue structures and/or content (e.g. regional hydration) status of mammalian bodies.

In an alternative use, RF energy at a specific frequency band can be passed through tissue of interest and the changes in amplitude of transmitted or reflected waves over time may be used for detecting the tissue structure or content changes. Additionally, phase difference between a transmitted sinusoidal RF wave and its reflection off the tissue can be used to detect the changes in tissue structure and content. For example, this phase difference is likely to increase or decrease with changing fluid density in the tissue because of the change in the bulk dielectric properties of the tissue.

Alternate forms of energy (e.g., terahertz EM signals, light, sound or mechanical waves) could be used similarly to detect tissue structure and content changes. Acoustic waves at frequencies (e.g. 1 kHz-100 kHz) lower than typically used for ultrasound have some useful properties that could be utilized for tissue structure and content change. These low frequency acoustic waves have much higher depth of penetration than ultrasound and may be built using cheap portable hardware.

Infrared light has been used in a variety of diagnostic equipment (e.g. pulse oximeters) for measuring parameters such as pulse rate, oxygen saturation etc. One of the main problems with infrared radiation or with terahertz radiation is their very small depth of penetration. Hence they are only capable of measuring changes on the surface or just below the surface. Consequently, changes in the tissue structure or content over the first few layers of the tissue could be tracked using infrared light or terahertz radiation.

Alternatively, bioelectric impedance of the tissues of interest can be used to monitor the changes in tissue structure and content. Bio-electric impedance analysis uses sinusoidal electrical currents or voltages of frequencies (preferably but restricted to between 0 Hz-200 kHz) and measures the voltage drop across the tissue of interest to determine the impedance (resistive and reactive) of the tissue to the flow of current. This impedance measurement is used to track and monitor the changes in tissue structure and content.

### Definitions

Body—a portion or all of the torso, arms, legs, head or neck of a subject, including any or all of the exterior and/or interior of the body.

Bioparameter—a physical factor associated with a body or user able to be measured and quantified.

Subject—a mammal being measured using the method or devices of the invention.

The invention described herein includes methods and devices for determining the effective change in electrical (including but not limited to conductivity, permittivity, permeability), physical (including but not limited density, viscosity etc) or chemical (including but not limited Cl—, Na+, K+ ions) properties of one or more inspected regions of tissue and/or bodily fluids. Change in these properties may result from change in either the composition and/or relative proportional volume of various heterogeneous components that comprise a living mammalian body. This change in composition and/or relative volume results in a change in the time of flight of an applied electromagnetic, optical or acoustic signal through the inspected body region and/or a change in the power attenuation of the signal of the applied signal through this inspected body region. These changes in signal are useful to monitor relative changes in structures and/or content (e.g., hydration/edema) of dermal, transdermal or subdermal, or deep tissue regions of mammalian bodies. These regional changes may be used to calculate changes occurring to either body regions or to the entire body and may be derived from a plurality of regional measurements occurring at one or more body sites using noninvasive or implanted measurement devices.

These changes may be, but are not limited to, changes associated with: normal bodily functions or processes, e.g. growth or development of tissues or organs, detection or development of disease states (e.g. detection of hypertension), the growth of tumors, or the monitoring of therapeutic regimens upon the measured region or aspects of the region (e.g. allograft or transplanted organ rejection). In addition, changes may also include, but are not limited to, changes in the body or measured region related to changes in hydration status and/or blood loss. In a preferred form of the invention, one or more devices are implanted within the body and measure changes in regional fluid content by application of energy from the device to the surrounding tissue and the measurement of the tissue response to said applied energy. When said energy is electrical, the measurement of tissue electrical impedance at one or more applied electrical frequencies may be measured. In certain embodiments, the relative change may be calibrated to allow quantification of this change, either in size and/or composition of the inspected region or parts thereof.

In an embodiment of the invention, the measurement device (transmitter, sensors plus circuitry) can be affixed to a body site by means of adhesive or it can be fully implanted within the body. In alternate embodiments, the device or sensors may be may be located in part or in whole on the outside of the body, or they may be affixed to the body by means of straps, clothing items or by anchoring directly to or beneath the skin or held against the skin by hand or other supports. In still other embodiments, the measurement device is not in direct contact with the surface of the skin. The scope of the invention is not limited to any one body site or means of affixing the device to the body or adjacent to or not in direct contact with the body. In yet other embodiments of the invention, a portion of the measurement device may be implanted and a second portion may be located on the outside of the body. In such embodiments, the second portion may serve as a power supply to the implanted portion, e.g. induction of electrical current in the implanted portion via electromagnetic interaction. In still other embodiments, the implanted portion may transmit a signal that is received by the external second portion (or vice versa) allowing measurement of the body region. Alternatively, the second portion may serve to aid communication for the purpose of data retrieval and/or instruction delivery to the implanted portion on either a continuous or intermittent basis.

In some embodiments of the invention, a second device, e.g. a data collection and display unit, may be in communication with the measurement device. Such communication may be bi-directional and include optical, electromagnetic, mechanical, electrical, and/or acoustic means. In one such embodiment of the invention, the antenna and/or aspects of the transmission circuitry utilized for measurements within the measurement device may also be employed for communication of the data set, or mathematical transforms of the data set, to one or more data collection units. In still other embodiments of the invention, the communication may utilize the body or portion of the body as an antenna or as a necessary component for the communication, e.g. acoustic signals or ultra wideband signal propagation. In yet other embodiments, the communication may be between one or more measurement devices, either implanted or on the surface of the body, prior to communication with the data collection unit.

Comparator activities may be performed by systems located in the measurement device, located in the data collection unit, located in a remote data management system or collocated in any of these devices.

The monitoring period may extend from a relatively short period, e.g. fractions of seconds or minutes, to a more extended period, e.g. hours or days, dependent upon the purpose of monitoring and/or user acceptance. In alternative embodiments, the monitoring period may be periodic, e.g. for an hour or two distributed over a day or for a day or two distributed over months. The needs for such monitoring periods again may be set by the purpose of monitoring and/or user acceptance.

### Measurement Device

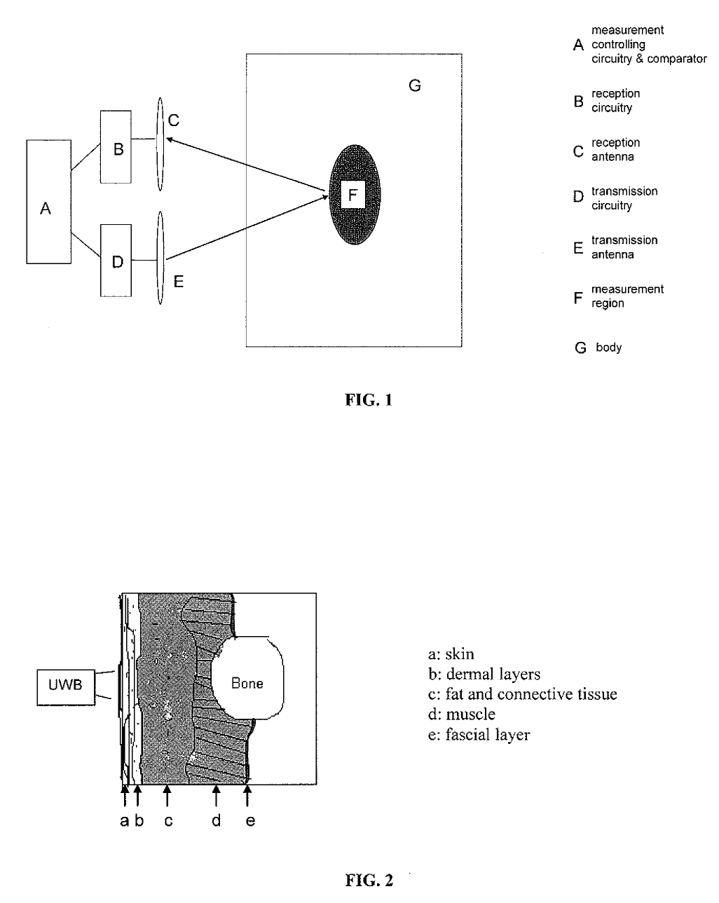

Ultra Wideband Radiation One form of electromagnetic wave that can be employed in embodiments of the invention is ultra wideband (UWB) radiation. However, the scope of the invention is not restricted to UWB but may also employ other forms electromagnetic waves, e.g. discrete frequency bands. The circuitry, power sources and transmission requirements of UWB are well known to those skilled in the art of radio electronics. One representation of components for the measurement device is shown in FIG. 1. As shown, a control element 10 is responsible for controlling the initiation of the electromagnetic wave signal, and the subsequent conversion of the reflection of the electromagnetic signal into a measurement data set. The control element 10 controls transmission circuitry 16 which transmits an input signal to a transmission antenna 18. The control element 10 may control various parameters of the transmitted UWB signal including the frequency, the duration, the power level as well as other parameters. The transmission circuitry 16 is electrically connected to the transmission antenna 18 which amplifies and directs the UWB signal to a measurement region 20 within a body 22. The measurement region 20 may be any general point of interest in any body. The transmitted signal 19 is shown reflecting off the measurement region 20 where the reflected signal 21 is then received by the reception antenna 14 and the reception circuitry 12. The control element also controls the reception circuitry 12.

In the example shown in FIG. 1, the transmitted signal 19 is shown to be reflected off of the measurement region 20. However, in some cases, the transmitted signal 19 may pass through the measurement region 20 and reflect off of another region, e.g., a bone. In addition, multiple reflected signals 21 may be received by the reception antenna 14 where the multiple reflections may be reflected off of multiple layers of tissue. The presence of a newly reflected signal at one time point that was not present in the data collected at a previous time point may be an indication of a new structure within the search region 20.

In addition to controlling the transmission circuitry 16 and the reception circuitry 12, the controlling element 10 also contains the comparator for determination of change in the measured region. Also shown are the corresponding signal transmission and reception circuit elements 16 and 12, respectively, as well as corresponding transmission and reception antennas 18 and 14, respectively, for the transmission and reception of said signals. A variety of arrangements of components may be employed in execution of the method of the invention, e.g. combined functionality within one circuit module or the use of a single transmission/reception antenna, and the method of this invention is not limited to any one method of execution. Likewise, in certain embodiments of the invention, a plurality of devices, or components, e.g. transmitters and/or receivers, may be employed to provide additional information of the region being measured.

UWB systems transmit signals across a much wider frequency than conventional systems. The amount of spectrum occupied by a UWB signal, e.g. the bandwidth of the UWB signal, can be about 25% of the center frequency or more. Thus, a UWB signal centered at 2 GHz could have a bandwidth of about 500 MHz and the bandwidth of a UWB signal centered at 4 GHz could be about 1 GHz. The most common technique for generating a UWB signal is to transmit pulses with durations less than 1 nanosecond.

Although with a UWB system, one can non-invasively and without touching the surface of the skin evaluate the gross anatomy internal organs of the body, in one preferred form of the invention, one or more UWB measurement devices are fully implanted within the body. UWB uses short pulses and reflections and the reflected signal strength and/or delay in signal time of flight of these pulses from different layers inside the body will provide data regarding anatomical structures, their location, dimensions, dielectric composition, and their movement within the body. Some typical applications of the UWB are organ movement or dimensional changes such as heart wall movement, measurement of heart wall thickness, kidney/liver/stomach, etc. dimensional change, respiration, and/or density changes as well as applications in obstetrics.

The main advantages of UWB over other technologies such as ultrasound are the following:
UWB signals do not need to be in contact with the skin because they are less affected by transmission through air than sound waves.
UWB signals do not attenuate in the bone and hence can obtain information inside cavities covered by bone, such as the brain.
UWB signals can be used to collect data through non-conductive material such as cloth, bedding, hazmat suits, or body armor.
UWB signals can be realized with a small number of inexpensive components enabling low power, portable applications.

As noted above, the UWB measurement system detects the changes in the structure of the tissue by examining signal reflections from the tissue layers. For example, a transmitter sends a UWB pulse and then receives the pulse reflections from the different layers of the tissue. Because the speed of the UWB radar is different in different types of tissues (e.g. the signal propagation is approximately 2.25 times faster in fat than in muscle) and the layers of tissue are at different depths, the reflections from the tissue layers reach the receiver at different times and have different amplitudes. Further, the attenuation of the signal as it travels through the tissue gives the density information of the tissue. For example, when placed next to a limb including a bone, as shown FIG. 2, the UWB radar measurement system 100 can assess any changes in fluid accumulation in the various layers by measuring the time delay of the received pulses and their amplitude relative to the corresponding parameters during baseline evaluation. The layers shown in FIG. 2 include a skin layer 1, a dermal layer 2, a fat and connective tissue layer 3, a muscle layer 4 and a fascial layer 5. Other layers may also be evaluated.

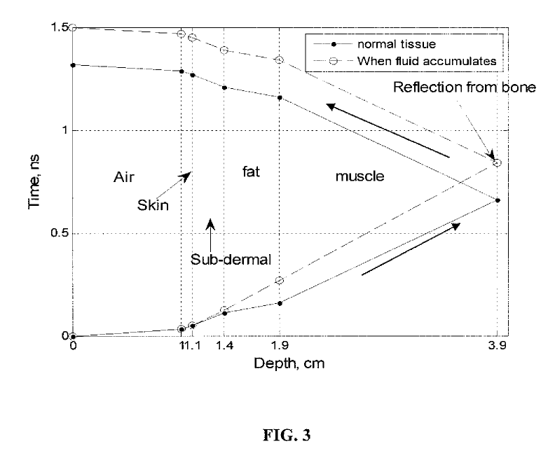

FIG. 3 illustrates an example of how a UWB pulse travels through tissue and how the fluid accumulation, for example, in the tissue affects the amplitude and delay of the reflected pulses. This is because fluid (e.g., water) has much higher permittivity than muscle and fat resulting in slowing of the pulse in the presence of fluid. Additionally, due to higher attenuation in saline, there are amplitude changes in the reflected waves. In generation of this simple model, it is assumed that the properties of fat and muscle approach those of saline as the influx of fluid into the cavity increases. In a certain embodiment of the invention, the extent of hydration change within a body region is assessed by a change, e.g. degree of attenuation of the UWB signal, the in reflection or transmission of the signal from transmitting antenna to a least one receiving antenna.

As shown in FIG. 3, a first baseline measurement is made and is depicted as a transmitted signal 34A and a reflected signal 34B. The transmitted signal 34A starts at a position 1 cm above the skin (listed as a depth of 0.0 on the horizontal axis). The signal 34A is shown passing through a skin layer from 1.0 cm to about 1.1 cm, a sub-dermal layer from about 1.1 cm to abut 1.4 cm, a fat layer from about 1.4 cm to about 1.9 cm, and a muscle layer from about 1.9 cm to about 3.9 cm, where the transmitted signal 34A is shown reflecting off of the bone layer resulting in the baseline reflected signal 348. The reflected signal 34B then passes through the other layers and experiences various delays and attenuations based on the type of material in each layer. It should be noted that FIG. 3 shows only one reflected signal, but this is done for purpose of clarity and skilled technologists will recognize that multiple reflected signals may be received and analyzed.

The baseline reflected signal 34B has a time of flight of about 1.3 nanoseconds to progress through the different layers from transmission to reception. At a later time, a second measurement is made and a transmitted signal 32A is directed into the same region for a subsequent measurement. The transmitted signal 32A is then reflected off the bone layer and is received by the measurement system 100. In this second measurement, the transmitted signal 32A and the reflected signal 32B are delayed substantially compared to the baseline transmitted signal 34A and reflected signal 34B. In this case the round trip time of flight is about 1.5 nanoseconds for the signals 32A and 32B compared to 1.3 nanoseconds for the baseline signals 34A and 34B. As discussed above, this may be due to the presence of more water and/or saline in the various layers. In addition to the delay in the signal, the amplitude of the signal may also be different (not shown in FIG. 3) which may also be analyzed to identify possible sources of attenuation such as saline as discussed above.

As discussed above, other signal forms may also be used in the measurement system 100, depending on the embodiment. Other signal forms that may be used by the measurement system 100 to evaluate the structure and/or content of tissues will now be discussed.

High Frequency Electromagnetic Radiation: Electromagnetic radiation at high frequencies, such as terahertz radiation, or higher, e.g. infrared light, could potentially be used to monitor changes in superficial tissue structure and content. This may be achieved by monitoring the amplitude of reflected radiation from the tissue surface. Any changes in the structure or content (edema) can be detected because of absorption by water in the tissues.

Acoustic Radiation; Although acoustic waves are mechanical waves, similar wave propagation principles could be used to detect changes in tissue structure and content. The speed of the acoustic wave in any material is related to temperature, the elastic properties of the material, and the material's density. Thus, any changes in the constituents of the tissue due to its changing physiology or morphology that are reflected by a change of its properties can be detected by using acoustic waves. In one embodiment, the invention comprises equipment configured to focus acoustic waves comprising frequencies in a range from about 1 kHz to about 100 kHz (high bandwidth short pulses or high bandwidth longer time signals composed of multiple frequencies like “chirps”) on tissues and monitor the transmitted and the reflected waves from the tissue. Depending on the type of application, three different methods of analyses are possible, a) Wave attenuation while passing through the tissue; b) Attenuation of reflected waves; and c) Phase difference between transmitted and reflected waves after reflection of a boundary, such as between bone and tissue. Additionally, any temperature changes inside the tissue due to inflammation or an infection response could be tracked because the speed of sound changes with temperature.

The method and devices for applying acoustic signals to one or more regions of the body are well known to those skilled in the art of acoustic signal generation and interpretation, including sonography. In a certain embodiments of the invention, the acoustic sensors are implanted within the body, thereby avoiding the need for acoustic coupling aids, e.g. gels, to ensure good contact with the body and the acoustic measurement device.

Bioelectric Impedance Signals: In another embodiment of the invention, as noted before, bioelectric impedance of the tissues of interest can be used to monitor the changes in tissue structure and content. This is achieved by passing electrical current through the tissue of interest and measuring the voltage drop across the tissue (or by exciting the tissue with a sinusoidal voltage and measuring the current through the tissue) and calculating the impedance of the tissue to the current flow. Changes in the amplitude of the measured signal as well as the phase difference between the voltage and current signals depend on the tissue properties. As the tissue structure changes (e.g., due to scar tissue growth, tumors etc) and/or content changes (e.g., due to edema, fat/muscle ratio etc), the amplitude of the impedance as well as the phase with respect to the current or voltage excitation change and can be monitored. Additionally, DC signals could also be used for obtaining the changes in the resistance of the tissue. For bio-electrical impedance, focusing of energy (or controlling the volume of measurement) may be achieved by changing the geometry of the electrodes used for driving the current and measuring the voltage. The method and devices for delivering and receiving electrical signals to one or more regions of the body, including necessary electrodes and circuitry, are well known to those skilled in the art of bioelectrical impedance.

In certain embodiments of the invention, the electrodes supplying and measuring the electrical signal are fully implanted in the body region of interest and the impedance change between one or more sets of electrodes. Such impedance measurements may employ a two point electrode arrangement, e.g. where current electrodes are the same as measurement electrodes, a four point electrode arrangement, e.g. where current electrodes are distinct from measurement electrodes, or a combination of two and four point arrangements. In addition, a plurality of electrodes may be employed enabling assessment of electric impedance vectors in a plurality of directions.

General Use: The measurement and storage of energy pulses or signals of any frequency comprise a data set which may include reference to time of measurement and/or location of measurement. Such measurement and storage activities may include transformation of the raw data. Such transformations may be useful, allowing facilitated storage of the data set, e.g. data compression, or otherwise facilitate transmission and/or analysis of the data set by the comparator. Signals for use by the comparator may be from one or more of applied energy sources, e.g. radiofrequency, acoustic, electrical or optical. In addition, these signals may be utilized in various combinations over time to provide greater insight into dynamically changing body regions or tissues, e.g. inspection of suspected tumors may be first registered using forms of radiofrequency energy detecting the presence of tissue of differing density. Subsequent observations may include impedance measurements to gauge the increased swelling, blood flow or edema around this site to more accurately provide a trend analysis of change over time.

In some embodiments of the invention, fiducial marker aids, signal alignment aids and/or signal improvement aids may be employed. These aids may include the use of mapping of body regions using electromagnetic signals or other techniques, e.g. MRI, to establish points of reference within data sets or provide aids to more precisely position the measurement device on the body. Employment of these points of reference thereby improves alignment of data sets enabling change in the target region to be more precisely determined. In alternate forms, these aids may include the use of passive or active devices affixed to or implanted within the mammalian body. These aids may provide reference signals or otherwise serve as landmarks to target the measurement device and/or data set. The fiduciary aids may include, but are not limited to: optical alignment aids, e.g. tattoos; signal reflective aids, e.g. implanted metal reflectors or conductive inks; inductively charged implanted radiofrequency transceivers; or implanted acoustic transmitters. Such aids may be arranged in geometric patterns, e.g. cross-hatched, to improve both interpretation of on-body position, e.g. signal alignment, and signal complexity in a known fashion through a three dimensional space to aid subsequent comparator activities.

In certain other related embodiments of the invention, materials either implanted or positioned about the inspected region, may be utilized to aid in the measurement process. For instance, these materials may be utilized to focus the electrical/electromagnetic/acoustic waves through regions of interest or may serve as a highly reflective target behind the region of interest, thereby increasing the effective signal strength of the applied electrical/electromagnetic/acoustic waves.

In other embodiments of the invention, the transmitter elements of the measurement device may have an identity assigned to it. Such identity may include the ability to determine antenna geometries and transmission frequencies. Likewise, other portions of electronic circuitry may have additional identities assigned to the remaining components of the circuitry. Such identities may be useful for enabling construction of disposable and reusable assemblies within the device and allow tracking of said assemblies. Also, such identities allow subsequent identification of the use of the device and form of the device in managing the data sets and coordinating findings of the comparator to the individual measured subject. In addition, such identities may have use in the assignment of encryption keys or other needs for secure transmission of information and assignment/display of the measured data sets.

### Comparator

The comparator subsystem of the control element 10 of FIG. 1 includes both a storage means and a means to determine change between data sets. These determinations may include the use of input threshold values, threshold set points determined by change from baseline value (or representation of one or more data points indicative of a baseline value). Alternatively, such comparator functions may include the use of rolling or moving averages to determine trends in the data set and to allow adjustment for data taken at different points within the day, e.g. diurnal variation adjustment. Still other forms of comparator activity may review populations or groups of data for the determination of initial baseline values and for trends of data sets or groups. The results of such comparator activity may be displayed graphically, e.g. showing baseline values and relative change from these values over time, including the projection of future trends.

In addition, the comparator subsystem may incorporate other factors, such as input parameters associated, e.g. weight, height, age, gender, disease status and medication history, fitness level, body site of device application, ethnicity, etc. or parameters derived from algorithms arising studies allowing further definition of change and/or the magnitude of such change. Such parameters may include factors relating subjective user or clinician perception of change to the measured bioparameter, either upon the event or periodically, e.g. daily.

In yet other embodiments of the invention, the comparator subsystem may include other factors including data derived from other measured bioparameters such as levels of circulating hormones or metabolites or activity measurements, or data obtained from environmental sensors, e.g. relative humidity, ambient temperature, etc. This invention may employ combinations of these as well as other factors and the scope of the invention is not limited to those factors and mathematical routines described herein.

In one or more embodiments of the invention, the comparator may analyze the measured data from one or more devices using one or more sensors to remove noise, motion artifacts, or other non-desired factors from the received data to enable determination of change in the measured region. Such measured data may include data collected over a period of time, e.g. seconds, minutes or days, or from one or more body locations. Such analysis may remove rapid noise factors, e.g. motion associated with body activity, or long term trends, e.g. habitual (eating) noise or diurnal shifts in one or more parameters. In related embodiments, measurements may be taken on either regular or irregular points in time to aid in the reduction of noise and/or predictable factors, e.g. data associated with meals or other predictable activities. Also, the system may learn through pattern analysis or be programmed to adjust the times and frequency of measurements to reduce noise and/or optimize power consumption of one or more of the measurement devices.

In certain embodiments, measured data from one sensing means, e.g. UWB, may be employed in conjunction with measured data from a second sensing means, e.g. impedance. Such use may include the use of one data set to aid in the calibration or adjustment of the second data set or to provide an additional factors from which the comparator may evaluate possible change.

The comparator subsystem may reside in a variety of locations. In one embodiment of the invention, the comparator may be contained either in part or in whole within circuitry necessary to acquire the data set. In other embodiments of the invention, the comparator may be located in a separate unit connected by wires to the sensors and/or sensor circuitry, e.g. the transceiver. In such embodiments, the sensors or sensor circuitry may have identities separate and distinct from the unit comprising in part or in whole the comparator activities. In still other embodiments of the invention, the location of comparator activities may shift in order to facilitate data analysis, e.g. to accommodate greater sophistication and/or larger data sets, or for other purposes, e.g. power management of devices, data collection units, etc.

In yet other embodiments of the invention, measured values or mathematical transforms, e.g. averages, or percentage change, of one or more measured bioparameters are transmitted through wireless means to a separate unit not necessarily located on the body. This separate unit may contain either in portion or entirely the appropriate elements and circuitry, e.g. transmission means, data storage and mathematical calculator functions and routines, to perform the comparator activities. Additional forms and locations for the comparator are readily conceivable and the scope of the invention is not limited to those described herein.

Upon determination of a change in status in the measured region, as well as the possible determination of the magnitude of such events, the comparator may be instructed to display such events to the subject, caregiver or third party individual. Display of any changes may also include the notification of no change as compared to all or a portion of the data set. Such displays may include visual displays, e.g. anatomical maps of the region including two dimensional and three dimensional representations, blinking or multicolored lights, numeric indices, graphs, or charts, audible sounds or mechanical forms, e.g. vibrations. Such displays may be located on the on-body measurement device, a local data collection unit or at a remote location connected to either the on-body measurement device or a local data collection unit by wireless or wired means.

In addition to possibly displaying the change, the comparator may store the event description, including date/time, magnitude and user identification, in a data storage. Such data storage may include electronic memory, magnetic tape or disk memory, optical memory or other form of retrievable memory. Such data may be retrieved from storage either on command or periodically from the memory storage. In certain embodiments, such retrieval may be through wireless means, e.g. infrared or radio frequency based data transmission or in other embodiments, such retrieval may be through wired means, e.g. by use of a docking station attached to a computer or by serial cable linkage to a computer.

In embodiments of the device involving closed-loop therapy management, the comparator communicates with an effecting mechanism, which acts to initiate a treatment protocol or control an already existing treatment protocol, or initiate a sequence of pre-specified actions. In certain embodiments, the effecting mechanism comprises a set of computer instructions programmed into a microprocessor. This effecting mechanism can share the resources with the sensing mechanism or it can have separate/additional resources. For example, when using bioelectric impedance method to sense tissue structure and content changes and using electrical stimulation as treatment, the sensing and stimulation electrodes can be the same. Similar arrangements can be made in cases when the sensing and effecting mechanisms use similar forms of energy (electrical, electromagnetic, acoustic, chemical, optical etc.).

In certain applications, the sensor energy, e.g. UWB or acoustic, may be utilized to wirelessly transmit information with another device and/or with comparator circuitry/logic This is advantageous in certain embodiments of the invention since the same core circuitry may be employed for both sensing and transmission thereby reducing component count, overall device size and minimizing costs. In such applications, the same core circuitry can be switched between antennas and other structures responsible for detecting physiological parameters and one or more other antennas, electrodes or other transmission structures used for data transmission. In some cases, the same antenna and/or related structures could be used for both data transmission and sensing if orientation and other factors allow for the overlapping use.

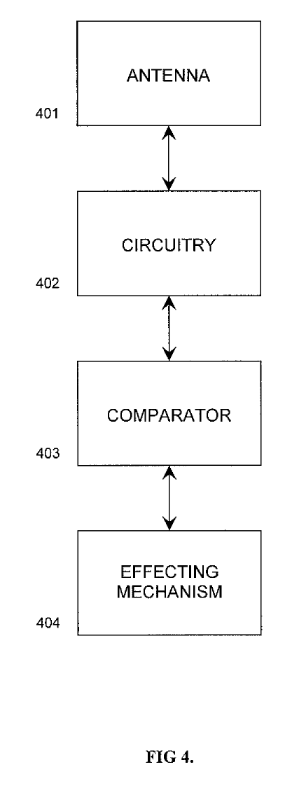

In certain embodiments of the invention, the effecting mechanism can be implemented as a computer program embedded within the device itself or in external programmable circuitry that communicates with the device through a wireless or wired means. In response to information or data from the comparator, the effecting mechanism acts to initiate treatments or actions for the purpose of ultimately causing a desirable change in the data being monitored. Referring now to FIG. 4, in one embodiment the system may comprise an antenna 401 which is in communication with programmable circuitry 402. The antenna and programmable circuitry 402 can be configured to transmit and receive electromagnetic wave signals. The circuitry 402 or other circuitry (not shown) can then convert said signals into one or more data sets which are then presented to a comparator 403 for performing mathematical calculations. This permits the determination of change in the tissue or hydration status in a measured region of the body. The system may also comprise an effecting mechanism 404 for effecting a desired change in the body region.

For example, the comparator may receive data consistent with an increase of fluid in the pulmonary space in a patient. This information is then communicated to an effecting mechanisms, e.g. an effector, which may initiate a number of desirable therapeutic actions, detailed in further below. The effector can also be programmed to initiate alarms, pages, short message service (SMS) text messages, audible sounds, lights, etc. to notify attendant personnel of any relevant change in status. Additionally, the effector can also record the actions it initiates or changes in a log such as on computer disk or onto paper through a printing device.

### Therapy Management

Initiated actions upon detecting a change in the tissue structure or density may include starting treatments, changing existing treatments, or initiating or changing pre-specified actions that reduce the deleterious effects of the changes detected. The effecting mechanism for initiating actions/treatments may include means to either direct the treatment towards the tissue where the change is detected or towards a different site of interest that has some causal effect over the observed tissue change detection area. In some embodiment of the invention, a measurement results in a therapeutic action for the body as a whole, e.g. the detection of regional fluid change (increase) resulting in a therapeutic response to increase diuresis. The effecting mechanism can also be directed to the initiation of obtaining more diagnostic information, e.g. triggering an automated chest x-ray or initiating a laboratory analysis to determine the creatinine level of an obtained blood sample.

For example, an electrical stimulation mechanism includes at least two electrodes placed such that the paths of current pass through the site of tissue change. Other corresponding mechanisms for delivery can be conceived for other forms of stimulation, e.g. antenna or waveguides for electromagnetic, light sources of the appropriate wavelengths for optical stimulation, heating elements for providing localized heat therapy, or acoustic stimulation and pumps for drug delivery. Treatment mechanisms that can be used, independently or in combination with each other, include, but not limited to, electrical stimulation (AC, DC, pulsatile currents), electromagnetic stimulation, optical stimulation (e.g. using frequencies known to elicit responses from certain cell types like mitochondria), acoustic stimulation (e.g. ultrasound), thermal stimulation (e.g. infrared heating, cooling, freezing), chemical stimulation (e.g. drug delivery or oxygen therapy), physical change (e.g. massaging to improve circulation or change in position), activation of an implanted device, or an implanted micro-chip or a MEMS based devices. Additionally, the action initiated may be a warning for the clinician to undertake an appropriate action, e.g. change of dressing, change of position, or change of medication. Drug delivery may include delivery of specific enzymes to facilitate other drugs (e.g. hyaluronidase for reducing scar tissue and improving absorption and dispersion) or a combination therapy such as using ultrasonic waves to breakup scar tissue around breast implants or other implanted devices or material.

For example, the effecting mechanism can be linked to an electronically controlled infusion device, infusing the drug furosemide, a loop-diuretic helpful in treating acute pulmonary edema. In response to data from the comparator, the effecting mechanism may then adjust the infusion device to increase the dosage rate of furosemide according to a schedule predetermined by the patient's physician. The effecting mechanism may also be programmed to slow the rate of other IV infusions it is electronically linked to, such as any saline infusions that are provided to the patient merely as a maintenance dose in order to regulate the total volume of fluids being supplied to the patient to a desired rate. After the effector mechanism has initiated its action(s), the comparator mechanism continues to collect and process data. This data is further presented to the effector mechanism which may again initiate or change actions based on parameters set by the patient's physician.

In some embodiments, the data from the comparator is communicated to more than one effecting mechanism which may initiate similar or completely different types of actions. For instance, one effecting mechanism may change drug dose as described above, while another may provide for further diagnostic testing or sampling to occur (e.g. turn on an X-ray machine to obtain a chest x-ray). In some embodiments, these effecting mechanisms are combined.

The effecting mechanism may also be partly or wholly controlled by a physician or other caregiver. In some such embodiments, the caregiver is notified of data set changes and in response controls at least some aspects of the effecting mechanism to control at least some aspects of the therapeutic response to those changes that is then implemented by the effecting mechanism.

### Use and Applications

In use, the measurement device or devices may be implanted, affixed to the subject or positioned about the subject and the device activated by means of a switch or other form of activation. The activation may take place prior to implanting or affixing the measurement device to the user. Such activation may also include the use of aids or other alignment tools to ensure correct positioning of measurements. Activation may yet further include activation by means of a switch or other means of a local data collection unit in wireless communication with the measurement device. In such embodiments, an identifier, e.g. a code or serial number, may be used to identify the measurement device to the data collection unit. Such identifiers may include further identifiers detailing the specification of one or more of the measurement sensors. Such identifiers may be transmitted automatically to the data collection unit upon activation or in alternate applications, may be input manually into the data collection unit.

In other embodiments, a single data collection unit may be in communication with a plurality of measurement devices located on or associated with more than one user. In such embodiments, the activation may include an identification means allowing identification of the user in addition to identification of the measurement device.

Upon activation, the measurement device may periodically or upon command obtain measured data from one or more body regions. The monitoring period per data set may be seconds, e.g. every second, or longer, dependent upon the nature of the bioparameter being measured for change. In other embodiments, the monitoring frequency may automatically adjust, dependent upon the rate of change in the desired portion of the measured region. In addition, different devices may have different configurations of sensors and/or different monitoring frequencies applied to the same subject. The measured data may be supplied directly to the comparator for analysis or it may be processed in some form prior to being supplied to the comparator for determination of change and/or magnitude of such change.

Applications of electromagnetic or other forms of energy waves for monitoring of change of one or more regions of a mammalian body include, but are not limited to:
Monitoring for change in body hydration and/or electrolyte levels over time, including detection of detrimental levels of systemic hydration, projection of future hydration status and recovery or return to acceptable levels of fluid and/or total ion composition levels of bodily fluids.
Monitoring for jugular vein distension associated with hypertension such as period measurements separated by intervals shorter than the determined heart rate to permit assessment of the magnitude of vascular distension (change) due to blood pressure.
Monitoring of internal organs, including heart, kidney or liver for changes such as fluid infiltration/local edema associated with organ failure and/or allograft rejection.
Monitoring of one or more organs, including heart for changes that indicate irregular, acute and/or chronic conditions, e.g. atrial fibrillation, acute renal failure, hyper or hypotension, and loss of consciousness.
Monitoring of ovarian development/folliculogenesis during the menstrual cycle to aid in the determination of proper timing for the administration of drugs associated with fertility and/or pregnancy.
Monitoring of wounds and/or scar tissue associated with wounds to aid in the detection of infection, impaired healing or to guide timing of wound treatments, e.g. debridement or for wound staging to determine the extent of deep tissue injury.
Monitoring of body locations, e.g. sacrum, hips or heels, to detect changes in underlying tissue fluid status associated with the pre-emergence of ulcers or other forms of skin/tissue disease states.
Monitoring of fluid build-up or change in internal body compartments, organs or muscle groups, e.g. internal hemorrhage or compartment syndrome, associated with disease state, trauma or surgical interventions to allow more effective detection and subsequent therapeutic response.
Detection of fluid build-up over time associated with the onset and/or progression of cardiogenic or non-cardiogenic pulmonary edema.
Detection of body composition change, e.g. change in muscle composition associated with wasting diseases such as HIV disease progression or muscular dystrophy caused by neuromuscular disorders.
Monitoring stenosis or occlusion of blood vessels, or implanted vascular devices e.g. venous grafts.

Additional applications may also include providing a user of the device with quantitative feedback regarding the magnitude of the measured change and any periodic nature to this change, e.g. time of day, such that the user may self-medicate in order to relieve the symptoms or otherwise take some form of therapeutic action associated with the change in the underlying bioparameter. Alternatively, the use of remote data management systems receiving data from one or more data collection units may permit clinician adjusted therapy changes from a remote location upon review of the data sets and output of comparator activities.

While the above detailed description has shown, described, and pointed out novel features of the invention as applied to various aspects, it will be understood that various omissions, substitutions, and changes in the form and details of the device or process illustrated may be made by those skilled in the art without departing from the scope of this disclosure. As will be recognized, the invention may be embodied within a form that does not provide all of the features and benefits set forth herein, as some features may be used or practiced separately from others. The scope of this disclosure is defined by the appended claims, the foregoing description, or both. All changes which come within the meaning and range of equivalency of the claims are to be embraced within their scope.

What is claimed is:

1. A method of effecting change over time in a body, the method comprising the steps of
determining change over time in one or more measured regions of the body using a plurality of data sets obtained by analysis of applied electromagnetic waves to said region, and
effecting an action using an effecting mechanism which results in desired further changes over time to said region.

2. The method of claim 1, wherein the comparator is at least partially implanted in the body.

3. The method of claim 1, wherein the electromagnetic waves are comprised of ultra wideband radio signals.

4. The method of claim 1 where the form of electromagnetic waves are comprised of signals of frequency bands other than ultra wideband radio signals.

5. The method of claim 1 wherein determining said change comprises determining a change in tissue structure.

6. The method of claim 4, wherein determining the change in tissue structure comprises detecting one or more of scar tissue growth, tumors, and fractures.

7. The method of claim 1 wherein determining said change comprises determining a change in tissue content.

8. The method of claim 6, wherein determining said change in tissue content comprises determining one or more of a fluid change, a hypo or hyper hydration, an edema, and a relative change in the amount of fat, muscle, connective tissue, or bone in said region.

9. The method of claim 1, wherein said the action controls a step in treatment protocol.

10. The method of claim 1, wherein the action controls a diagnostic tool.

11. The method of claim 1, wherein the action controls the delivery of a drug.

12. The method of claim 1, wherein the determined change is a change in transplant organ status.

13. The method of claim 1, wherein the determined change is change in ovary dimension.

14. The method of claim 1, further comprising employing fiducial points or alignment aids.

15. A system for effecting change over time in tissue structure and content in a body region, the system comprising:
antenna and circuitry configured to transmit and receive electromagnetic wave signals;
circuitry configured to convert said signals into one or more data sets;
a comparator subsystem for performing mathematical calculations on at least two data sets for the determination of change in the tissue or hydration status of said measured body region; and
an effecting mechanism for effecting a change in the tissue over time in said body region, wherein said effecting mechanisms communicates with said comparator subsystem.

16. The system of claim 15, wherein said antenna and circuitry configured to transmit and receive electromagnetic waves are fully implanted in the body.

17. The system of claim 15, wherein said effecting mechanism is configured with a predetermined treatment protocol.

18. The system in claim 15, wherein said effecting mechanism initiates an action in response to communications from said comparator subsystem.

19. A method of effecting change over time in one or more measured regions of the body using a plurality of data sets obtained by analysis of applied acoustic waves to said region.

20. The method of claim 19, wherein the form of acoustic waves are comprised of signals in a range between 1 Hz and 100 kHz.

21. The method of claim 19, wherein determining said change comprises determining a change in tissue structure.

22. The method of claim 21, wherein determining the change in tissue structure comprises detecting one or more of scar tissue growth, tumors and fractures.

23. The method of claim 19, wherein determining said change comprises determining a change in tissue content.

24. The method of claim 23, wherein determining the change in tissue content comprises determining one or more of a fluid change, hypo or hyper hydration, an edema, and relative changes in an amount of fat, muscle, connective tissue or bone in said region.

25. The method of claim 19, wherein said change is associated with a healing wound.

26. The method of claim 19, wherein said change is associated with compartment syndrome.

27. The method of claim 19, wherein said change is a change in transplant organ status.

28. The method of claim 19, wherein said change is a change in ovary dimension.

29. The method of claim 19, further comprising employing fiducial points or alignment aids to target said signals.

30. A system for effecting change over time in tissue structure and content in a body region, said system comprised of:
antenna and circuitry for the transmission or reception of applied acoustic wave signals;
circuitry for converting said signals into one or more data sets;
a comparator subsystem for performing mathematical calculations on at least two data sets for the determination of change in the tissue or hydration status of the measured body region; and
an effecting mechanism for effecting a change in the tissue over time in said body region, wherein said effecting mechanisms communicates with said comparator subsystem.

31. The system of claim 30, wherein said antenna and circuitry for the transmission or reception of applied acoustic wave signals is fully implanted in the body.

32. The system of claim 30, wherein said effecting mechanism is configured with a predetermined treatment protocol.

33. The system in claim 30, wherein said effecting mechanism initiates an action in response to communications from said comparator subsystem.

34. A method of effecting change over time in one or more measured regions of the body using a plurality of data sets obtained by analysis of bio-electric impedance of said body region.

35. The method of claim 34, further comprising performing electrical tissue excitation including a plurality of frequencies between 0 Hz (DC) and 1 MHz.

36. The method of claim 34, wherein effecting said change comprises determining a change in tissue structure.

37. The method of claim 36, wherein effecting said change in tissue structure comprises detecting one or more of scar tissue growth, tumors, and fractures.

38. The method of claim 34, wherein effecting said change comprises determining a change in tissue content.

39. The method of claim 38, wherein effecting said change in tissue content comprises detecting one or more of a fluid change, hypo or hyper hydration, an edema, and relative changes in amount of fat, muscle, connective tissue or bone.

40. The method of claim 34 where said change is associated with a healing wound.

41. The method of claim 34, wherein said change is associated with compartment syndrome.

42. The method of claim 34, wherein said change is a change in transplant organ status.

43. The method of claim 34, wherein said change is a change in ovary dimension.

44. The method of claim 34, further comprising employing fiducial points or alignment aids.

45. A system for effecting change over time in tissue structure and content in a body region comprised of:
electrodes and circuitry configured to transmit and receive electrical signals;
circuitry for converting said signals into one or more data sets indicative of a measure of bio-electric impedance of said body region;
a comparator subsystem for performing the mathematical calculations on at least two data sets for the determination of change in the tissue or hydration status of the measured body region; and
an effecting mechanism for effecting a change in the tissue over time in said body region, wherein said effecting mechanisms communicates with said comparator subsystem.

46. The system of claim 45, wherein said electrodes and circuitry configured to transmit and receive electrical signals are fully implanted in the body.

47. The system of claim 45, wherein said comparator is located in a separate data collection unit in wireless communication with said electrodes and circuitry.
